(19) United States
(12) Patent Application Publication
(10) Pub. No.: US 2026/0363260 A1
(43) Pub. Date: Nov. 19, 2026

(54) BACTERIA

(71) Applicant: Chr. Hansen A/S, Hoersholm (DK)

(72) Inventors: Paula SZYMCZAK, Farum (DK); Thomas JANZEN, Broenshoej (DK); Rute NEVES, Hoersholm (DK)

(73) Assignee: Chr. Hansen A/S, Hoersholm (DK)

(21) Appl. No.: 19/693,717

(22) Filed: Jun. 1, 2026

Related U.S. Application Data
(62) Division of application No. 17/259,792, filed on Jul. 15, 2019.

(30) Foreign Application Priority Data
Jul. 13, 2018 (EP) EP18183416.9

(51) Int. Cl. A23C 9/123; A23C 19/032; C12N 1/205; C12N 15/10; C12R 1/46

(52) U.S. Cl. CPC A23C 9/1238; A23C 19/032; C12N 1/205; C12N 15/1024; A23C 2220/202; C12R 2001/46

(57) ABSTRACT

The present invention relates to novel S. thermophilus strains that are not attacked by common phages and novel Streptococcus strains with improved stability against phage attacks, methods to obtain Streptococcus strains with improved phage resistance, their use in the manufacture of fermented milk-based products, and novel milk based products containing the strain.

### CROSS-REFERENCE TO RELATED APPLICATIONS

This application is a divisional of U.S. application Ser. No. 17/259,792 filed on Jan. 12, 2021 which is a U.S.C. 371 national application of international application no. PCT/EP2019/068980 filed on Jul. 15, 2019 which claims priority or the benefit under 35 U.S.C. 119 of European application no. 18183416.9 filed Jul. 13, 2018 the contents of which are fully incorporated herein by reference.

### SEQUENCE LISTING

This application contains a Sequence Listing in computer readable form. The contents of the electronic sequence listing created on May 20, 2026, named SQ.XML and 15, 157 bytes in size, is hereby incorporated by reference in its entirety.

### FIELD OF INVENTION

The present invention relates to novel Streptococcus strains that are not attacked by common phages, its use in the manufacture of fermented milk-based products, and novel milk based products containing the strain. In a related aspect, the present invention relates to methods for improving phage robustness and cell count stability of Streptococcus such as Streptococcus thermophilus.

### BACKGROUND OF INVENTION

Fermented milk products, such as fermented milk drinks, lactic acid bacteria beverages, yoghurt, cultured milks and cheese, are often produced by providing milk substrates, especially based on animal milks, such as cow milk, goat milk, sheep milk and the like, as culture media and fermenting the medium with lactic acid bacteria. During the production of a fermented milk product, phages may attack the bacteria, leading to a reduction in the viable cell count of the lactic acid bacteria.

In particular fermented milk products which are consumed as health-promoting foods having beneficial physiological effects, such as intestinal function controlling effect and immunopotentiating effect, are dependent on a high number of viable bacteria. Production of fermented milk products comprising probiotic bacteria of the species Streptococcus thermophilus is often challenged by phage attacks in the dairies, resulting in a product with lowered content of viable bacteria. It is however difficult to replace a strain susceptible to phage attack by another strain of the same species, as the replacement strain seldom has a similar functionality, such as acidification activity, flavor profile and/or the same probiotic character.

Streptococcus thermophilus is one of the most common bacteria used worldwide as a starter in the production of fermented foods, such as cheese and yoghurt (Binetti, Quiberoni, and Reinheimer 2002; Mahony et al. 2016). This microorganism is a thermophilic, aerotolerant, Gram-positive coccus, and a member of a heterogeneous group of lactic acid bacteria (LAB) (Lahtinen et al. 2012). The extensive use of S. thermophilus in dairy plants, through cultivation in large vats, has resulted in increased susceptibility to bacteriophage infections (Labrie, Samson, and Moineau 2010b). Indeed, phage outbreaks represent the major cause of slow or faulty fermentations, frequently leading to a lower quality of dairy products and suboptimal production costs.

Diverse treatments have been applied to minimize phage infections in the dairy environment. Predominant approaches include chemical and physical methods for equipment sanitation (Guglielmotti et al. 2012), as well as a culture replacement and strain rotation programs (Derkx et al. 2014). The latter require strains with identical technological performance, but different phage sensitivities (Binetti, Bailo, and Reinheimer 2007). Thus, isolation and characterization of bacteriophage insensitive mutants (BIMs) of strains used in dairy starter cultures has been widely performed.

Several methods for generating BIMs of S. thermophilus have been proposed. The strategies include insertional mutagenesis (Lucchini, Sidoti, and Brüssow 2000), the secondary culture method (Binetti, Bailo, and Reinheimer 2007), serial passaging in the presence of high phage titers (Mills et al. 2007), chemical mutagenesis (Rodríguez et al. 2008) or transformation with an antisense mRNA-generating plasmid (McDonnell et al. 2018). Generally, the acquired resistance is due to the activation of intracellular resistance mechanisms, mainly clustered regularly interspaced short palindromic repeat (CRISPR)-Cas systems or restriction-modification (R-M) systems (Lucchini, Sidoti, and Brüssow 2000; Binetti et al. 2007; Mills et al. 2010). Additional phage resistance systems, such as abortive infection (Abi) (Larbi et al. 1992) or superinfection exclusion (Sie) (Ali et al. 2014), have also been detected in S. thermophilus. However, those mechanisms probably are not very widespread and therefore, they do not commonly mediate resistance in BIMs of dairy strains.

The mode of action of CRISPR-Cas and R-M systems is similar in the way that they both target specific genetic sequences of the invading phage (Dupuis et al. 2013). CRISPR and cas genes provide adaptive immunity that uses sequence memory to target incoming DNA (Barrangou and Horvath 2017). Restriction enzymes of R-M systems recognize and cleave foreign DNA at defined sites within the recognition sequence, while the host DNA is resistant to cleavage due to modifications at these sites (Guimont, Henry, and Linden 1993; Pingoud et al. 2005). Although CRISPR-Cas and R-M systems were reported to work together for generating the efficient phage resistance in S. thermophilus (Dupuis et al. 2013), the activation of only one of them may result in BIMs that are partly phage sensitive (Deveau et al. 2008). Phages commonly acquire point mutations in their genomes to overcome bacterial immunity mediated by the intracellular systems (Barrangou et al. 2007; Paez-Espino et al. 2013, 2015; Deveau et al. 2008; Labrie, Samson, and Moineau 2010a). Additionally, phages evolved particular anti-restriction strategies, such as the acquisition of a methylase gene (McGrath et al. 1999), and anti-CRISPR strategies like the production of proteins that inhibit CRISPR-Cas activity (Joe Bondy-Denomy et al. 2013; Joseph Bondy-Denomy et al. 2015). Therefore, the dynamic and unstable nature of R-M and CRISPR-Cas systems suggests that BIMs whose resistance is mediated by those mechanisms may not be suitable for industrial applications (Mahony et al. 2016).

To obtain robust phage-resistant variants of S. thermophilus strains, strategies were proposed to select for BIMs whose phage resistance was mediated by mechanisms independent of R-M or CRISPR-Cas systems. For example, BIMs with inhibited phage adherence to the bacterial cell walls were selected by immunoprecipitation in flow cytometry (Viscardi et al. 2003). Resistance in these BIMs results either from modification or masking the phage receptor structure (Labrie, Samson, and Moineau 2010a; Seed 2015). Thus, understanding the interactions between the antireceptors of phages and their receptors present on the cell surface of a S. thermophilus bacterial strain is a determining factor to develop phage-resistant cultures.

Knowledge on structure and properties of bacterial cell walls is advantageous when studying components recognized by phages. The cell wall of Gram-positive bacteria consists of a peptidoglycan (PG) layer that surrounds the cytoplasmic membrane and that is decorated with other glycans and proteins (Chapot-Chartier and Kulakauskas 2014).

The cell wall glycans comprise two groups of cell surface associated polysaccharides:
(i) exocellular polysaccharides including exopolysaccharides (EPS) that are secreted into the extracellular matrix and capsules (CPS) that are in most cases covalently bound to PG and form a thick outer layer surrounding cells (Zeidan et al. 2017) and
(ii) polysaccharides interpolated with PG (WPS), such as pellicles (Chapot-Chartier et al. 2010) or rhamnose-containing cell wall polysaccharides (RGP) (Mistou, Sutcliffe, and Van Sorge 2016).
(iii) The third group of cell wall glycans comprises teichoic acids, classified into wall teichoic acids (WTA) that are covalently bound to PG (Brown, Santa Maria Jr., and Walker 2013) and lipoteichoic acids (LTA) that are anchored to the membrane (Reichmann and Gründling 2011). Genes encoding biosynthesis of cell surface associated glycans are typically organized in clusters (Zeidan et al. 2017; Brown, Santa Maria Jr., and Walker 2013;

Reichmann and Grundling 2011) and different glycans can share the same component, namely undecaprenyl phosphate as a lipid carrier (Chapot-Chartier and Kulakauskas 2014). Proteins, which are synthetized in the cytoplasm, can be either incorporated into the membrane or linked to the bacterial cell wall through different modes of attachment (Chapot-Chartier and Kulakauskas 2014).

In lactic acid bacteria, the cell wall components involved in the interactions between bacteria and their phages are best studied in Lactococcus lactis (Mahony et al. 2016). A correlation between the receptor type present on the cell surface and the tail-tip morphology of the phage has been established (Mahony and van Sinderen 2012). Members of two dominating groups of L. lactis phages, 936 and P335, recognize specific oligosaccharides of the highly variable cell surface associated polysaccharides (Ainsworth et al. 2014; Bebeacua, Tremblay, et al. 2013; Collins et al. 2013; Mahony et al. 2013). Those phages possess complex baseplate structures at the end of the tail (Bebeacua, Tremblay, et al. 2013; Collins et al. 2013; Bebeacua et al. 2010; Spinelli et al. 2006). L. lactis phages from the c2 group have a small tail tip (Lubbers et al. 1995) and use proteins, either PIP or YjaE, to act as receptors for the irreversible interaction with their hosts (Geller et al. 1993; Derkx et al. 2014). For another dairy bacterium, Lactobacillus delbrueckii, LTA were designated as receptors for the phage LL-H (Räisänen et al. 2004, 2007) and they interact with a fiber located at the end of the phage tail (Munsch-Alatossava and Alatossava 2013).

To date, four main types of S. thermophilus phages have been characterized (Szymczak et al. 2017; McDonnell et al. 2017, 2016). The two dominating groups, the cos- and pac-containing phages, are distinctive on a genetic level, but they display similar morphological characteristics. They possess long tails (typically more than 200 nm in length) with or without fibers on the tail tip (Mahony and van Sinderen 2014; Szymczak et al. 2017; McDonnell et al. 2017). Phages belonging to the group 5093 have tails of similar length as the cos- and pac-containing phages, but terminate with globular baseplates (Mills et al. 2011). The group 987 comprises phages with short tails (120 to 150 nm in length) and complex baseplate structures (Szymczak et al. 2017; McDonnell et al. 2016), which are genetically related to L. lactis phages from P335 group (Labrie et al. 2008).

The comparative analysis of S. thermophilus phage genomes has confirmed that this population can be divided into the previously defined groups cos, pac, 5093, and 987. Considering the growing number of phages of the groups 987 and 5093, which also use pac and cos DNA packaging mechanisms, the conventional classification of S. thermophilus phages based on DNA packaging mechanisms (cos and pac) and structural protein composition may be misleading.

Therefore, without limiting the scope of present invention, the inventors of present invention propose new names for the two dominating groups: the pac group to be described as group O1205, because phage 01205 was the first pac-group representative defined, and the cos group to be described as group DT1, because phage DT1 was used as a model of cos-group phages in several studies. The novel nomenclature will be more accurate in reflecting the current grouping of S. thermophilus phages. Hence it is understood herein that the pac-group is intended to comprise the 01205 group and the cos-group is intended to comprise members of the DT1 group.

As for other dairy bacteria, the receptors of phages on the cell surface of S. thermophilus May be cell wall associated polysaccharides, teichoic acids or proteins. Presence of CPS was reported to increase phage sensitivity in S. thermophilus strains (Rodríguez et al. 2008), while loss of ropy phenotype was associated with the acquisition of phage resistance in a non-CRISPR BIM (Mills et al. 2010). The identity of phage receptors in S. thermophilus still remains largely elusive. Hence, identifying phage receptors of S. thermophilus will contribute to generating future strategies that aim in designing robust phage-resistant dairy starter cultures.

### SUMMARY OF INVENTION

An objective of the present invention is to provide a method to obtain phage resistant mutants of strains of Streptococcus thermophilus with improved phage insensitivity and robustness. The method and mutants disclosed herein may further provide for increased phage insensitivity when compared to conventional CRISPR-cas or R-M facilitated methods as evidenced by e.g. the Heap Lawrence assay.

The present inventors have found that mutagenesis in genes related to glycan biosynthesis results in phage resistant strains which could replace the mother strain in fermented dairy products. As discussed above, conventional phage hardening methods based on intracellular mechanisms provide a limited protection against phages. By conferring the resistance by extracellular mechanisms i.e. phage attachment, increased phage insensitivity is provided.

The present inventors have provided genetic and biochemical evidences that cell wall glycans mediate phage-host interactions in S. thermophilus. To that end they identified mutations in a range of putative receptor mutants generated from industrial S. thermophilus strains, visualized phage-host interactions using super-resolution structured illumination fluorescence microscopy, and performed biochemical assays to identify the macromolecules recognized by phages with different antireceptor structures. This is the first report on the identity of phage receptors at the bacterial cell surface of S. thermophilus and their influence on phage attachment and phage insensitivity.

By applying the herein disclosed knowledge on bacterial phage receptors and phage-host interactions, methods to prevent phage infections in dairy plants are provided.

### Aspects of the Invention

In a first aspect, the present invention relates to a method for the manufacture of a phage resistant mutant of a strain of the species Streptococcus thermophilus, said method comprises:
mutating a culture of the strain (the mother strain);
optionally exposing the mutated strains to a phage that attacks the mother strain
selecting a phage resistant mutant comprising a mutation in a gene involved in glycan such as e.g. extracellular polysaccharide (EPS) or capsular polysaccharide (CPS) biosynthesis or rhamnose-containing cell wall polysaccharides (RGP) synthesis.

Aspect 2. A method for manufacture of a cell count stabilized mutant of a strain of the species Streptococcus thermophilus, said method comprises:
subjecting a culture of the strain to mutagenesis (the mother strain);
optionally exposing the mutated strains to a phage that attacks the mother strain
selecting a phage resistant mutant comprising a mutation in a gene involved in glycan such as e.g. extracellular polysaccharide (EPS) or capsular polysaccharide (CPS) biosynthesis or rhamnose-containing cell wall polysaccharides (RGP) synthesis.

Aspect 3. The method according to any preceding aspects, wherein the strain from which the mutant is derived is selected from the group consisting of STCH_09 (DSM19243), STCH_12 (DSM32826), STCH_13 (DSM32841), STCH_14 (DSM21408) or STCH_15 (DSM32842) or mutants or variants of any of these.

Aspect 4. A method according to any of aspect 1 to 3 wherein the gene involved in glycan such as e.g. extracellular polysaccharide (EPS) or capsular polysaccharide (CPS) or rhamnose-containing cell wall polysaccharides (RGP) biosynthesis is a glycosyltransferase.

Aspect 5. A method according to any of the preceding aspect wherein the gene involved in glycan biosynthesis has at least 90% such as e.g. 95%, such as e.g. at least 98%, such as e.g. at least 99%, such as e.g. 100% sequence identity to one or more of the sequences SEQ ID NO: 1, SEQ ID NO: 2, SEQ ID NO:3, SEQ ID NO:4, SEQ ID NO: 5 or SEQ ID NO: 6.

Aspect 6. A method according to any of the preceding aspects wherein the mutagenesis comprises a substitution, deletion or insertion of one or more nucleotides in one or more of the sequences SEQ ID NO: 1, SEQ ID NO: 2, SEQ ID NO:3, SEQ ID NO: 4, SEQ ID NO: 5 or SEQ ID NO: 6.

Aspect 7. A method for providing a phage resistant and/or cell count stabilized mutant of a strain of the species Streptococcus thermophilus said method comprises:
introducing a mutation (e.g. by means of genetic engineering) in one or more of the genes encoded by the sequences SEQ ID NO: 1, SEQ ID NO: 2, SEQ ID NO: 3, SEQ ID NO:4, SEQ ID NO: 5 or SEQ ID NO: 6 wherein said mutation results in a change or impairment in the glycan such as e.g. extracellular polysaccharide (EPS) or capsular polysaccharide (CPS) or rhamnose-containing cell wall polysaccharides (RGP) biosynthesis.

Aspect 8. A mutant of a strain of the species Streptococcus thermophilus obtained by the method of any of aspects 1-7.

Aspect 9. A strain of the species Streptococcus thermophilus, wherein the expression of a protein encoded by a sequence having at least 98%, such as e.g. 99%, such as e.g. 99.9% sequence identity to at least one of the sequences SEQ ID NO: 1, SEQ ID NO: 2, SEQ ID NO: 3, SEQ ID NO:4, SEQ ID NO: 5 or SEQ ID NO: 6 is impaired and wherein said mutation results in a change in glycan such as e.g. extracellular polysaccharide (EPS) or capsular polysaccharide (CPS) or rhamnose-containing cell wall polysaccharides (RGP) biosynthesis.

Aspect 10. A strain of any of aspects 8 or 9, wherein said strain shows increased robustness against phage attacks, e.g. by impairing attachment of phages to the cell surface.

Aspect 11. A strain according to aspect 10 wherein the phage is of the cos-pac- or 987 type.

Aspect 12. Use of a strain of the species Streptococcus thermophilus of any of aspects 8 to 10 for fermenting a milk substrate.

Aspect 13. A bacterial culture, such as a starter culture, which contains at least 10E8 CFU per gram of a mutant of a strain of any of aspects 8 to 11.

Aspect 14. The bacterial culture of aspect 13, which is in frozen or dried form, such as freeze dried or spray dried.

Aspect 15. A kit comprising the strain of any of aspects 8 to 11 or a bacterial culture of aspects 13 or 14, wherein the kit further comprises cryoprotectants and or germination booster components.

Aspect 16. A food product containing a strain of any preceding aspects, which is a fermented milk product, such as a drinking yogurt, a cheese or a yogurt.

### DEFINITIONS

In the present context, the term “resistant to phage” refers to the lactic acid bacterium strain is able to propagate (at optimal growth temperature) in milk which contains 1000 phages per ml, i.e. the bacterium is able to reach a cell density above 10E8 cfu/ml after 48 hours when inoculated at a concentration of 10E5 cfu/ml. Cfu is “cell forming units”.

The term “cell count stability” should be understood as a measure of the amount of colony forming units (CFU) per gram product. The higher Streptococcus thermophilus CFU/g over time, the more cell count stable is the tested strain. The cell count stability may be measured using the pour plate method.

The objective of present invention is to make a strain where the phage receptor, such as glycan (e.g. EPS/CPS or RGP) is altered to inhibit phage attachment and by this inhibit phage propagation by the strain, when compared to a corresponding parent or wild-type strain. As explained below it is routine work for the skilled person to make such a strain when equipped with the teachings disclosed herein. For instance, by introducing a stop codon or a frame shift insertion in the phage receptor biosynthesis gene, which could give a non-functional gene that would e.g. either express no phage receptor or express a partial length inactive phage receptor. Alternatively, a mutation could be made in a promoter, or the gene, which e.g. could give a phage receptor variant that has some activity but which for all herein related practical objectives is essentially inactive. A way to measure the inactivity of the phage receptor is simply to analyze the bacterium for increased resistance to a suitable representative panel of different bacteriophages. As explained below this is routine work for the skilled person and if the bacterium as described herein has a substantial increased resistance to the panel of bacteriophages then it is herein understood that the phage receptor is essentially inactive. Inactivation of the phage receptor does generally not negatively affect viability, growth rate or acid production of the LAB. See working examples herein.

The term “essentially inactive” should be understood in relation to the objective of the present invention, wherein the objective is to (essentially) inactivate a gene or regulatory element which is essential for the synthesis of a phage receptor, e.g. a gene associated with glycan biosynthesis, more specifically a glucosyltransferase involved in the synthesis of glycan.

Other genes involved in phage resistance can (essentially) be inactivated in line with the above methods.

The term “phage receptor” denotes a molecule which is synthesized by the collective action of biosynthetic proteins. An example of such enzyme is glycosyl transferase, which may be encoded by a sequence having at least 90% such as e.g. 95%, such as e.g. 96%, such as e.g. 97%, such as e.g. 98%, such as e.g. 99%, such as e.g. 100% sequence similarity to one or more of SEQ ID NO: 1-6 disclosed herein.

The term “phage receptor genes” are genes encoding proteins that are involved in synthesis of cell surface components such as glycans, more specifically “phage receptor genes” may mean sequences having at least 90% such as e.g. 95%, such as e.g. 96%, such as e.g. 97%, such as e.g. 98%, such as e.g. 99%, such as e.g. 100% sequence similarity to one or more of SEQ ID NO: 1-6 disclosed herein.

By the expression a “phage receptor is functionally inactive with respect to phage infection” is referred to e.g. that a bacterium which carries a phage receptor gene coding for said mutated phage receptor has improved resistance to at least one bacteriophage.

The term “improved resistance to a bacteriophage” denotes that the bacteria strain when tested in e.g. a plaque assay, such as the assay described as “Determination of phage resistance by the agar overlay method” or the “Heap Lawrence assay” described below have an improved phage resistance to at least one phage e.g. expressed as the difference in pfu/ml (plaque forming unit per ml) obtainable with said at least one bacteriophage on the given strain, compared to the pfu/ml obtainable with the same bacteriophage on the parent strain. A strain with improved resistance to a bacteriophage preferably show a reduction of pfu/ml of a factor at least 50, such as at least 100, e.g. 500, preferably at least 1000, more preferably at least a factor 10000 or more.

### Methods to Essentially Inactivate the Phage Receptor

As discussed above, it is routine work for the skilled person to make a strain as described herein, where the phage receptor is essentially inactive, e.g. by introducing a mutation in a phage receptor gene.

It is routine work for the skilled person to choose an adequate strategy to e.g. introduce a suitable modification of the phage receptor gene in order not to get expression of an active phage receptor.

One may randomly mutagenize (e.g. by UV radiation or chemical mutagenesis) and select for mutations wherein the phage receptor is essentially inactive. Further one could select for relevant spontaneous mutations, wherein the phage receptor is essentially inactive. Alternatively, one may use protein engineering (PE) techniques to introduce mutations to render the phage receptor gene dysfunctional and hence inhibit synthesis of the phage receptor.

In a preferred embodiment the phage receptor is inactive.

### Methods to Assay Protein Inactivation

As said above, a way to measure the inactivity of the phage receptor is simply to analyze the bacterium for increased resistance to bacteriophages.

Routinely this may be done by use of a standard plaque assay. The plaque assay evaluates the phage resistance of a strain of interest as the difference in pfu/ml (plaque forming units per ml) obtainable with a given bacteriophage on the strain of interest, compared to the pfu/ml obtainable with the same bacteriophage on the parent strain.

Accordingly, a lactic acid bacterium as described herein may be characterized by that it has improved resistance to the bacteriophage and/or improved cell count stability.

Preferably, the lactic acid bacterium as described herein has improved resistance to the phage deposited according to the present invention.

Alternatively, the strain genome or parts thereof may be sequenced. An alternative way to measure the inactivity of the receptor is to analyze the corresponding receptor gene sequence to see if it comprises a suitable modification that cause e.g. an inactivation of the gene. As explained above a suitable modification may be many things such as a stop codon, an insertion that e.g. cause frame shift, a deletion, a mutation etc. It is routine for a skilled person (e.g. by sequencing the gene) to identify if the gene comprises such a suitable modification.

Accordingly, in a preferred embodiment the lactic acid bacterium as described herein comprises a suitable modification in the gene, wherein the modification results in that essentially no active protein is expressed and no receptor is synthesized. More preferably, the modification results in that no active protein is expressed.

A further way to measure the inactivity of the receptor is to analyze if active receptor is present in the membrane of the bacterium. This may be done by a standard isolation method as described in working examples herein.

Accordingly, in a preferred embodiment the lactic acid bacterium as described herein does not comprise measurable amount of active receptor in the membrane.

As used herein, the term “lactic acid bacterium” designates a gram-positive, microaerophilic or anaerobic bacterium, which ferments sugars with the production of acids including acetic acid, propionic acid and lactic acid as the predominantly produced acid. The industrially most useful lactic acid bacteria are bacteria of the species Lactobacillus and Streptococcus, and are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product. Such cultures are in general referred to as “starter cultures” or “starters”.

In the present context, the term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder. Preferably, at least part of the protein in the milk substrate is proteins naturally occurring in milk, such as casein or whey protein. However, part of the protein may be proteins which are not naturally occurring in milk. Prior to fermentation, the milk substrate may be homogenized and pasteurized according to methods known in the art.

The term “milk” is to be understood as the lacteal secretion obtained by milking any mammal, such as cows, sheep, goats, buffaloes or camels. In a preferred embodiment, the milk is cow's milk. The term milk also comprises milks derived from plant material, such as soy milk. Optionally the milk is acidified, e.g. by addition of an acid (such as citric, acetic or lactic acid), or mixed, e.g. with water. The milk may be raw or processed, e.g. by filtering, sterilizing, pasteurizing, homogenizing etc., or it may be reconstituted dried milk. An important example of “bovine milk” according to the present invention is pasteurized cow's milk. It is understood that the milk may be acidified, mixed or processed before, during and/or after the inoculation with bacteria.

The expression “fermented milk product” means a food or feed product wherein the preparation of the food or feed product involves fermentation of a milk base with a lactic acid bacteria. “Fermented milk product” as used herein includes but is not limited to products such as thermophilic fermented milk products, e.g. yoghurt, mesophilic fermented milk products, e.g. sour cream and buttermilk, as well as fermented whey and cheese products.

The term “fermented milk drink” is a drinkable product obtained by fermentation of a milk substrate with lactic acid bacteria, such as bacteria of the species S. thermophilus. The product may be drinkable from a cup or a bottle, or via a straw. The product may be homogenized, e.g. after fermentation.

“Homogenizing” as used herein means intensive mixing to obtain a soluble suspension or emulsion. If homogenization is performed prior to fermentation, it may be performed to break up the milk fat into smaller sizes so that it no longer separates from the milk. This may be accomplished by forcing the milk at high pressure through small orifices.

“Fermentation” in the methods of the present invention means the conversion of carbohydrates into alcohols or acids through the action of a microorganism. Preferably, fermentation in the methods of the invention comprises conversion of lactose to lactic acid. Fermentation processes to be used in production of fermented milk products are well known and the person of skill in the art will know how to select suitable process conditions, such as temperature, oxygen, amount and characteristics of microorganism(s) and process time. Obviously, fermentation conditions are selected to support the achievement of the present invention, i.e. to obtain a fermented milk product.

In the present context, the term “packaging” (a suitable amount of) the fermented milk in a suitable package relates to the final packaging of the fermented milk to obtain a product that can be ingested by e.g. a person or a group of persons. A suitable package may thus be a bottle or similar, and a suitable amount may be e.g. 10 ml to 5000 ml, but it is presently preferred that the amount in a package is from 50 ml to 1000 ml.

In the present context, the term “mutant” should be understood as a strain derived from another strain (mother strain) by means of e.g. mutagenesis, radiation and/or chemical treatment, and/or selection, adaptation, screening, etc. The term also includes mutants with improved or altered phage resistance, e.g. phage hardened mutants or mutants showing improved cell count stability. It is preferred that the mutant is a functionally equivalent mutant, e.g. a mutant that has substantially the same, or improved, properties (e.g. regarding yield, viscosity, gel stiffness, mouth coating, flavor, post acidification, acidification speed, and/or phage robustness) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 1000, no more than 100, no more than 20, no more than 10, or no more than 5, treatments are carried out. In a presently preferred mutant, less than 5%, or less than 1% or even less than 0.1% of the nucleotides in the bacterial genome have been changed (such as by replacement, insertion, deletion or a combination thereof) compared to the mother strain.

In the present context, the term “variant” should be understood as a strain which is functionally equivalent to a strain of the invention, e.g. having substantially the same, or improved, properties e.g. regarding viscosity, gel stiffness, mouth coating, flavor, post acidification, acidification speed, sedimentation, probiotic activity, and/or phage robustness). Such variants, which may be identified using appropriate screening techniques, are a part of the present invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1 BIM Formation and Property Testing

### Materials and Methods

### Bacteria, Phages, and Growth Conditions

Streptococcus thermophilus strains and phages used for this study are listed in Table 1.

TABLE 1
List of S. thermophilus strains and phages from
the Chr. Hansen A/S collection used in this study

| | | Dairy | Sensitive | |
|---|---|---|---|---|
| Strain | Characteristics | production | towards phage | Phage group |
| STCH_09 | Fast-acidifying | Fermented | CHPC1057 | pac-containing |
| | strain with a | milks | | |
| | texturizing | | | |
| | phenotype | | | |
| STCH_13 | Fast-acidifying | Cheese | CHPC1014 | cos-containing |
| | strain | | | |
| STCH_14 | Fast-acidifying | Cheese | CHPC1046 | cos-containing |
| | strain | | | |
| STCH_15 | Fast-acidifying | Cheese | CHPC926 | 987 |
| | strain | | | |

Strains were stored at −40 °C in growth medium supplemented with 15% (wt/vol) glycerol and cultured overnight at 37 °C in LM17 broth (M17 broth [Oxoid, Denmark] with 2% [wt/vol] lactose) or anaerobically at 37 °C on LM17 agar plates (M17 agar [Oxoid] with 2% [wt/vol] lactose). If the bacterial cells were used for tests with phages, the growth medium was additionally supplemented with 10 mM CaCl₂) and 10 mM MgCl₂ (LM17-Ca/Mg). Streptococcus pneumoniae strain Pen6 (Filipe and Tomasz 2000), which was used as a control for the procedure of subtracting cellular components, was stored at −80 °C in growth medium supplemented with 15% (wt/vol) glycerol and cultured in a casein-based semisynthetic medium C+Y at 37 °C as described before (Garcia-Bustos, Chait, and Tomasz 1988). Phages were propagated as previously described in (Szymczak et al. 2017) and stored at 4 °C. Phage titers as well as the host ranges of investigated phages with bacterial strains were determined by using the double agar overlay spot test, as described before (Kropinski et al. 2009). Following overnight incubation under the appropriate growth conditions, the PFU per milliliter were calculated.

Bacteriophage insensitive mutants (BIMs) were formed using two methods. The plating method was adapted from the published protocol (Mills et al. 2007), where an overnight culture of a sensitive host was mixed with adequate phages at multiplicity of infection ≥1 (MOI, ratio of PFU to CFU per milliliter), plated in a soft top agar (LM17-Ca/Mg broth and agar mixed 1:1), and monitored for the appearance of phage-resistant colonies after 24-48 h of incubation under the growth conditions. If no colonies grew, MOI was reduced by mixing bacteria with a diluted phage lysate, and the procedure was repeated. For increasing the probability that the generated BIMs would acquire unique mutations, several single colonies of each wild type (WT) were inoculated into individual tubes and plated on separate plates after mixing with adequate phages. Due to the inefficient lysis with one of the phages used in the study, the secondary culture method was performed (Binetti, Bailo, and Reinheimer 2007), where LM17-Ca/Mg broth was inoculated with 1% overnight culture of a sensitive host, followed by addition of adequate phages at MOI=10 or MOI=0.01 and incubation at 37 °C. Survival cells were collected at two time points, after 5 h and 72 h of incubation, centrifuged at 15,000 g for 10 min, re-suspended in saline, mixed with adequate phages at MOI≥1, plated in a soft top agar, and monitored for the appearance of phage-resistant colonies after overnight incubation under the growth conditions. BIMs generated in both assays were purified by streaking on LM17 agar plates and incubating under the growth conditions in three sequential repetitions.

### Volumetric Pipette Viscosity Test

To test differences in the exocellular polysaccharides (EPS/CPS) production between the strain with texturizing phenotype and its BIMs, 250 ml of boiled milk was inoculated with 1% overnight cultures and incubated overnight at 37 °C. Samples were cooled to room temperature, gently mixed, and pipetted with a 25 ml pipette. The time of an unforced flow through the pipetted was measured in three repetitions. Thresholds for viscosity changes were set as follows: 25-34 s (reduced viscosity), 35-44 s (normal viscosity), 45-54 s (increased viscosity).

### Adsorption Inhibition Assay

For determining phage inhibition by monosaccharides, the described procedure (Valyasevi, Sandine, and Geller 1990) with some modifications was followed. Either glucose, galactose, rhamnose or glucosamine, to a final concentration of 200 mM, was added to cultures at early-exponential phase (OD₆₀₀=0.2) and immediately inoculated with adequate phages (10⁶ PFU/ml). Prepared controls included: cultures without monosaccharides and without phages, without monosaccharides and with phages, with monosaccharides and without phages. Growth of bacteria was followed by measuring OD₆₀₀ every hour for 7 hours and after overnight incubation. Duplicate replication of the assay was conducted.

### Microscopy Techniques

Microscopy screening of overnight cultures was performed to detect changes in cell chain lengths between WTs and BIMs. Photographs were taken using a Zeiss Axioplan 2 microscope equipped with a Plan-Neofluar objective (100×/1.3 oil Ph3) and a Zeiss Axiocam 503 mono camera (Zeiss, Germany).

Presence of exocellular polysaccharides (EPS/CPS) was tested using the India ink negative staining technique (Pachekrepapol et al. 2017) with the modifications that a mix of 7 μl of India ink with 7 μl of fresh milk and 3 μl of bacterial sample was prepared on a microscopy slide. Following air-drying, samples were visualized under the Zeiss Axioplan 2 microscope, with the specifications described above. After acquisition, photos were processed with ZEN software (black edition, version 14.0.0.201).

Changes in phage adsorption to the bacterial cell walls before and after depletion of cellular components were visualized under a fluorescence microscope. Freshly propagated phages were mixed 1000:1 (vol/vol) with a 10-fold-diluted SYBR Gold stock solution (Invitrogen, USA) and incubated overnight in dark at 4 °C (Dupont et al. 2004; Szymczak et al. 2017). Bacterial cultures at exponential phase (OD₆₀₀=0.5) and samples obtained during purification of cellular fractions in steps no. 1-4 (Table 3) were mixed with stained phages at MOI≥10 in LM17-Ca/Mg. Mixtures were immobilized on a thin layer of 1% agarose in PreC medium (Henriques et al. 2013). Photographs were taken with a 1 s exposure time using a Zeiss Axio Observer microscope with a Plan-Apochromat objective (100×/1.4 oil Ph3). Images were acquired with a Retiga R1 CCD camera (QImaging, Canada) using Metamorph 7.5 software (Molecular Devices, USA). After acquisition, images were processed using ImageJ software (Abràmoff et al. 2004). Phage binding patterns were visualized by super-resolution structured illumination microscopy (SR-SIM), due to its improved resolution compared to conventional microscopy (Monteiro et al. 2015). Bacterial cultures at exponential phase (OD₆₀₀=0.5) were stained with 2 μl/ml Nile Red (Invitrogen), incubated for 5 min at room temperature with agitation in dark and washed twice with LM17-Ca/Mg. Membrane-stained bacterial cells were mixed with SYBR Gold-labeled phages (MOI≥10) and mounted on a 1% PreC agarose pad as specified above. Imaging was performed by Elyra PS.1 microscope (Zeiss) using 50% 561-nm laser with 50 ms exposure for Nile Red and 20% 488-nm laser with 50 ms exposure for SYBR Gold. Images were acquired using five grid rotations, with 34 mm grating period for the 561-nm laser and 28 mm for the 488-nm laser, followed by the reconstruction and processing with ZEN software (black edition, version 14.0.0.201).

Transmission electron micrograph images of phages were generated following the method described before (Szymczak et al. 2017).

### Depletion of Cellular Components

Purification of cellular fractions was performed with the previously described method (Carvalho et al. 2015), with a modification that overnight cultures were sub-cultured into 2 L of LM17 broth (for S. thermophilus) or 2 L of C+Y medium (for S. pneumoniae) with the initial OD₆₀₀=0.01 and grown until OD₆₀₀ was between 0.5 and 1.0 (step no. 1). Chemical and enzymatic treatments were applied on the bacterial cell walls to progressively remove different cell wall components. Briefly, boiling cells with sodium dodecyl sulfate (SDS), followed by washing with MiliQ water, was performed to obtain cells devoid of surface proteins, membrane and membrane proteins (step no. 2). The sample was probably devoid of exocellular polysaccharides (EPS/CPS) loosely associated with the cell surface, as they were detached in multiple centrifugations applied in this step. The subsequent treatment with enzymes, lithium chloride (LiCl), ethylenediaminetetraacetic acid (EDTA), and acetone was executed to remove components ionically bound to the cell wall, such as proteins, as well as lipoteichoic acids (LTA) and intracellular components, i.e. DNA and RNA (step no. 3). The obtained cell walls with wall teichoic acids (WTA), polysaccharides interpolated with cell walls (WPS) and EPS/CPS anchored to peptidoglycan (PG) were treated with 46% hydrofluoric acid (HF) according to the protocol (Carvalho et al. 2015) and incubated at 4 °C for 72 h (step no. 4). In this step, WTA and cell surface polysaccharides were separated from PG. Aliquots of the cellular fractions (Table 3) were stored at −20 °C until further analyzes were performed.

Monosaccharide composition in samples collected during purification of cellular fractions (Table 3) was examined using the high-performance anion-exchange chromatography coupled with pulsed amperometric detection (HPAEC-PAD) as described before (Carvalho et al. 2015). The volumes of samples used for hydrolysis with hydrochloric acid (HCl) prior to the injection in the column were: 100 μl for cells collected at exponential phase, 40 μl for cells devoid of surface enzymes, membrane and membrane proteins (this amount corresponded to about 1×10⁹ cell equivalents), 20 μl at concentration 40 mg/ml for purified cell walls, 20 μl at concentration 20 mg/ml for purified PG. Standards for monosaccharides typically present in cell walls of Gram-positive bacteria (glucosamine, N-acetylglucosamine, muramic acid, N-acetylmuramic acid, rhamnose, glucose, galactose, ribitol, fucose, ribose, mannose, glucuronic acid (Zeidan et al. 2017; Delcour et al. 1999b)) were eluted under the same conditions to enable identification of chromatogram peaks.

Muropeptides present in purified PG were prepared and analyzed by reverse phase HPLC as previously described (Carvalho et al. 2015).

To exclude BIMs that acquired phage resistance due to the activation of a CRISPR-Cas system, a colony PCR was performed with primers specific for three CRISPR locus in S. thermophilus (Horvath et al. 2008b). The PCR reactions were prepared using PCR Master Mix (Roche, Germany) with the following conditions: 94 °C×2 min, followed by 30 cycles of 94 °C×45 s, either 48 °C. (CR2 and CR3) or 51 °C (CR1)×45s, 72 °C×2 min, with a final extension of 72 °C×5 min. PCR products were visualized on a 1% tris-acetate-EDTA (TAE) agarose gel. To perform full genome sequencing, DNA of the selected S. thermophilus strains was isolated using the DNA DNeasy Blood and Tissue kit with the protocol for Gram-positive bacteria (Qiagen, Germany) and sent for sequencing on the Illumina MiSeq platform with 2×250-bp paired-end sequencing (Illumina, USA).

Sequencing reads were trimmed, analyzed, and assembled using CLC Genomics Workbench 10.1.1 (Invitrogen). The assembled contigs were annotated by RASTtk (Brettin et al. 2015). SNPs analysis of WTs and BIMs was performed with CLC Genomics Workbench 10.1.1 (Invitrogen). Detected mutations were further evaluated to exclude false hits, i.e. SNPs at the end of contigs, SNPs in non-coding regions not related to a putative promoter or a putative terminator, SNPs at mobile element proteins, SNPs not resulting in amino acids changes. The analysis was made using CLC Main Workbench 7.7.3 (Invitrogen). Revised mutations were additionally verified using PCR, followed by Sanger sequencing (Macrogen, The Netherlands).

### Heap Lawrence Assay

The phage resistance robustness may be assessed using a so-called Heap Lawrence assay as outlined below.

An overnight (ON) culture of the strains to be assessed (wild type plus BIMs) is made by inoculating 250 μL of 10% RSM with 50 μL stock culture and incubate ON at 42′C. The ON culture is then diluted 5 times with fresh 10% RSM, 50 μl diluted culture is added to MTP containing 650 μl 10% RSM. Cells are allowed to grow for 1 hour whereafter a high titer phage lysate was added. Strain and phage are incubated at 42 °C. Acidification is monitored after 6-8 hrs after which strains and phage are left ON. The next day MTPs are centrifuged at 4000 rpm for 10 mins and supernatant, containing the phages, is transferred to a greiner tube. Part of the supernatant is mixed 1:1 with phage lysate in a new tube. This phage-mix is then used as a source of phage in another round of Heap-Lawrence as described for day 2. The procedure is repeated for many cycles, thereby allowing the phages to adapt to overcome the strain's phage resistance. By monitoring the number of cycles it takes for a phage to become virulent (indicated by the inability of the strains to acidify the milk) an indication of the phage robustness is obtained.

### Results

Here we select for BIMs harboring receptor defects, thus, isolates that became phage-resistant due to the activation of a CRISPR-Cas system were excluded. To that end, a colony PCR was performed with primers specific for the CRISPR1, CRISPR2, and CRISPR3 loci in S. thermophilus (Horvath et al. 2008a). BIMs with spacer(s) acquisitions were visualized on an agarose gel as a product of a larger size compared to a WT (data not shown). In total, 67 out of 142 tested BIMs were rejected from the investigation as potential CRISPR mutants. The remaining BIMs were subjected to phenotypic assays intended to select candidates with effective phage resistance and with properties of receptor mutants, presumably associated with modifications in cell wall glycans. A spot test was used to assess reduction of phage titers in non-CRISPR BIMs. Mutants selected for sequencing did not form plaques with their adequate phages, which confirmed activation of phage resistance mechanisms independent from CRISPR-Cas systems. Deficiency of phage adhesion to non-CRISPR BIMs was tested by mixing BIMs with SYBR Gold-labeled phages and screening under a fluorescence microscope. BIMs of strains STCH_09 and STCH_15 had visibly reduced phage adsorption compared to the WTs (FIG. 1), which suggested modifications in cell surface components serving as phage receptors. Observations under a microscope were also useful to detect changes in chain length between WTs and their non-CRISPR BIMs. Mutants of strains STCH_14 and STCH_15 formed elongated chains compared to the WTs. This observation could indicate mutations in cell wall glycan synthesis machinery since wall teichoic acids (WTA) and cell surface associated polysaccharides are essential for the proper division of bacterial cells (Chapot-Chartier and Kulakauskas 2014; Wu et al. 2014; Brown, Santa Maria Jr., and Walker 2013; Mistou, Sutcliffe, and Van Sorge 2016). A volumetric pipette viscosity test was performed for the texturizing strain STCH_09 and its non-CRISPR BIMs to detect changes in formation of exocellular polysaccharides (EPS/CPS). Four out of seven BIMs of STCH_09 selected for sequencing had reduced or increased viscosity compared to the WT. Phenotypic data revealed diverse characteristics of generated non-CRISPR BIMs and aided in selecting for putative receptor mutants, i.e. effective non-CRISPR BIMs with inhibited phage adhesion and/or difference in chain lengths and/or changes in viscosity compared to the WT. From those, 31 mutants (from 3 to 10 per strain) were genome sequenced.

### Mutating Glycan Biosynthesis Genes Inhibits Phage Adsorption

Mutations in genes encoding glycan biosynthetic pathways were detected in the genomes of BIMs with properties of receptor mutants. The gene modifications are described in Table 2.

TABLE 2
List of mutations in genes encoding glycan biosynthetic pathways detected
in phage-resistant mutants (BIMs) generated in this study

| | | | | Amino | Phenotypic |
|---|---|---|---|---|---|
| | Mutated | Putative gene | Type of | acid | change |
| BIM | gene | function | mutation | position | compared to WT |
| STCH_09_BIM | epsH | glycosyltransferase | AA | 262 Thr | Reduced |
| | | | substitution | | adsorption, |
| | rgp | glycosyltransferase | Gene | 197 Gln | Enhanced |
| | | | truncation | | viscosity |
| STCH_13_BIM | epsD | tyrosine- | AA insertion | 160 | Not detected |
| | | kinase | | | |
| STCH_14_BIM | epsE | priming | Gene | 37 Gln | Long chains |
| | | glycosyltransferase | truncation | | |
| STCH_15_BIM_1 | epsE | priming | Gene | Whole | Reduced |
| | | glycosyltransferase | deletion | gene | adsorption, |
| | | | | | Long chains |
| STCH_15_BIM_2 | epsK | glycosyltransferase | AA | 75 Glu | Reduced |
| | | | substitution | | adsorption, |
| | | | | | Long chains |
| STCH_15_BIM_3 | | | Gene | 93 Trp | Reduced |
| | | | truncation | | adsorption, |
| | | | | | Long chains |
| STCH_15_BIM_4 | | | Gene | 82 Ser | Reduced |
| | | | truncation | | adsorption, |
| | | | | | Long chains |
| STCH_15_BIM_5 | | | Gene | 93 Trp | Reduced |
| | | | truncation | | adsorption, |
| | | | | | Long chains |

Amino acid residues: Threonine (Thr), Glutamine (Gln), Glutamic acid (Glu), Tryptophan (Trp), Serine (Ser), Isoleucine (Ile), Valine (Val), Lysine (Lys)

In brief, nucleotide substitutions that could lead to amino acid substitutions in two glycosyltransferases were detected in STCH_09_BIM, which was generated from the texturizing strain and was deficient of phage adsorption compared to the WT (FIG. 1a). The glycosyltransferase epsH belongs to the exocellular polysaccharide (eps) operon (Zeidan et al. 2017), while the other glycosyltransferase belongs to the rhamnose-containing polysaccharide operon (Yamashita et al. 1998). Mutations in genes of eps operons were also found in BIMs of fast-acidifying strains STCH_13, STCH_14, and STCH_15. Insertion of three nucleotides in the epsD gene of STCH_13_BIM led to introduction of an additional amino acid in the gene product, while nucleotide substitution in the epsE gene of STCH_14_BIM led to the gene truncation. A complete deletion of the glycosyltransferase epsE was observed in STCH_15_BIM_1. The four other mutants of STCH_15 had unique mutations in the glycosyltransferase epsK, which resulted either in amino acids substitution, as for STCH_15_BIM_2, or frameshift and nonsense mutations and ultimately nonfunctional proteins, as for STCH_15_BIM_3, STCH_15_BIM_4, and STCH_15_BIM_5. The BIMs of STCH_15 had either reduced or lack of phage adsorption observed under a fluorescence microscope (FIG. 1b).

In conclusion, the detected mutations could affect production of cell surface associated polysaccharides, which resulted in loss of phage adherence to some BIMs. Hence by mutating cell wall glycans that act as receptors for phages in S. thermophilus phage adsorption and sensitivity was decreased.

### Phage Localization

The fluorescent signals of phages CHPC926, CHPC951, and CHPC1057 were localized at division sites of the host cells: at the septum, in the areas where a septal membrane ring began to build or where the cell wall has been produced and split (FIG. 2b, panels 1-3). Phages CHPC1014 and CHPC1046, which are cos-containing phages that hold fibers at the end of their tails, displayed a dispersed fluorescent signal that was evenly surrounding the host cells (FIG. 2b, panels 4-5).

The type of adsorption and the phage tail-tip morphology could not be correlated. Phage adsorption in the septal areas of the cells was observed for the phage with baseplates as well as pac-containing phages with tail fibers, while dispersed adsorption was observed for cos-containing phages with tail fibers. Two adsorption patterns could indicate that different types of S. thermophilus phages recognize diverse cell wall structures or that location of the recognized macromolecules on the cell surface is strain dependent. The latter alternative seemed to be more plausible, based on the additional fluorescence microscopy observations made for strain STCH_12. This strain is sensitive towards several cos- and pac-containing phages, which seemed to adsorb to STCH_12 with the same, spotty pattern as pac-containing phage CHPC951 (data not shown).

Phages with different antireceptor structures attached to different cellular fractions of their hosts: phage CHPC951 adsorbed to the surface of strain STCH_12 until WTA, WPS and EPS/CPS anchored to PG were removed from the cell walls, while adsorption of phage CHPC926 to the surface of strain STCH_15 was reduced with the cells devoid of surface enzymes, membrane and membrane proteins and the phage-host interaction disappeared completely after cell wall proteins and LTA were depleted. Thus, a fiber on a phage tail presumably establishes a binding complex with one of the cell wall glycans, WTA or cell wall associated polysaccharides, but not with PG, while the phage with baseplates interacts either with cell wall proteins, LTA or EPS/CPS on the cell surface.

The results of this study provide genetic and biochemical evidences that cell wall glycans are involved in phage adsorption to S. thermophilus used in dairy industry. Phages bind to macromolecules that are either regularly distributed along the cell length or locate in septal areas of the cells. The latter type of adherence is mediated by different cell wall components, which depend on the phage antireceptor structures. Fiber-ended phages adsorb to polysaccharides anchored to peptidoglycan (WPS or EPS/CPS), while phages with baseplates form binding complex with exocellular polysaccharides (EPS/CPS) loosely associated with cell surface.

Phage-resistant mutants (BIMs) of S. thermophilus strains, generated in this study, held mutations in genes encoding glycan biosynthetic pathways, which serves as a genetic indication that cell surface glycans mediate phage adsorption to this species.

The results of this study show that strains with and without the texturizing phenotype acquire mutations in genes of the eps operon, as a response for phage infections. Moreover, mutations in glycan biosynthetic pathways occurred in BIMs generated with phages that belong to different groups. This includes the dominating pac- and cos-containing phages with a tail fiber as well as 987-type phages with baseplates at the tail tip. The putative role of cell surface associated polysaccharides as phage receptors was supported by the fact that mutations in glycosyltransferase genes were linked to the loss of phage adsorption to some of the generated BIMs. For the BIMs with unchanged phage adhesion, other parameters such as phage DNA injection may be compromised as a result of the change in EPS/CPS structures (Mills et al. 2010).

In conclusion, the results of this study provide evidence that cell wall glycans, other than PG, are involved in phage adsorption to S. thermophilus strains. The molecular factors mediating phage adherence to the septal areas of S. thermophilus are polysaccharides anchored to PG, as for a phage with a tail fiber, and CPS/EPS loosely associated with cell surface, as for a phage with baseplates. Identifying phage receptors of S. thermophilus will contribute to generating future strategies that aim in designing robust phage-resistant dairy starter cultures.

### Example 2

### Site Directed Inactivation of EPS/CPS Biosynthesis

This study was performed to confirm the role of glycans in a host recognized by S. thermophilus phages. To that end, an industrial S. thermophilus strain was genome edited to inactive EPS/CPS biosynthesis operon. Subsequently, acquisition of a phage-resistance in the generated mutants was confirmed.

### Materials and Methods

### Construction of Mutants Affected in EPS/CPS Biosynthesis

S. thermophilus STCH_15 was used to construct mutants with inhibited activity of the eps operon. Vector pGh9ΔepsE was designed by cloning a 535 bp fragment of epsE gene into the thermosensitive plasmid pG⁺host9 (Maguin, Prévost, Ehrlich, & Gruss, 1996). This gene is essential for the biosynthesis of EPS and CPS (Zeidan et al., 2017). The cloned fragment had 97% identity to the epsE gene fragment of S. thermophilus STCH_15. Vector pGh9ΔepsE was introduced to STCH_15 by electrotransformation with the conditions described before (Buckley, Vadeboncoeur, LeBlanc, Lee, & Frenette, 1999). After transformation, cells were recovered at 30 °C for 2 h and plated on LM17 plates containing 3 μg/ml erythromycin as a selective marker. Plates were incubated under anaerobic conditions at 30 °C for 3 days. Positive transformants were identified by a colony PCR with primers specific for pGh9ΔepsE vector.

Integration of the plasmid into the chromosomal DNA was obtained by growing positive transformants at 45 °C in the presence of erythromycin. Plasmid integration was confirmed by two PCR assays. The first PCR contained two primers binding upstream and downstream from the integration site. It was designed to deselect derivatives with wild-type (WT) genotype. The second PCR contained one primer specific for pGh9ΔepsE vector and another primer binding downstream from the integration site. This primer set was used to identify derivatives with integrated plasmid. The expected product sized for the two assays was 1000 and 1054 bp, respectively. The positive integration was confirmed by Sanger sequencing.

### Determination of Phage-Resistance of the Generated Mutants

Selected mutants with plasmid integration were tested to evaluate the effect of the introduced mutation on phage-resistance. Plaque assay with phage CHPC926 was performed as described before with the modification that erythromycin was added to the growth medium of mutants (Kropinski, Mazzocco, Waddell, Lingohr, & Johnson, 2009). Acidification in milk, i.e. 9.5% reconstituted skimmed milk boiled at 98 °C for 30 min, was measured by monitoring pH for 16 hours. Cultures were inoculated into two parallel tubes with milk supplemented with erythromycin when required. Phage CHPC926 was added to one of the tubes at the final concentration 10⁷ pfu/ml. Control tube without inoculum was prepared.

### Results

Strain STCH_15 was successfully transformed with vector pGhost9ΔepsE. Five colonies with positive plasmid integration into the chromosomal DNA were identified by the PCR assays and confirmed by fragment sequencing. Two of the generated mutants, termed STCH_15_ΔepsE_1 and STCH15_ΔepsE_2, were further characterized.

The two mutants were completely resistant towards phage CHPC926. Based on the plaque test, the introduced mutation resulted in 9-log reduction of the phage titer (no single plaques observed for the two mutants). As observed in the acidification test, STCH_15_ΔepsE_1 and STCH_15_ΔepsE_2 acidified milk at the same rate in the presence and absence of phages (FIG. 3). They initiated acidification after 1.5 h incubation under the growth conditions. The performance of the mutants was superior to the performance of STCH_15, which started acidifying milk after 2.5 h incubation. Acidification of the WT in the presence of phages was delayed for 3 h compared to the sample without phages added.

### CONCLUSIONS

In this study, an inactivation of epsE gene resulted in obtaining phage resistance towards the phage from group 987. The improved phenotype was obtained by genome engineering and without challenging WT with phages. The results of this study confirmed that glycans, such as polysaccharides synthetized by the eps operon, are phage receptors of the phage from group 987. This observation established the general role of glycans as cell surface receptors recognized by S. thermophilus phages.

### DRAWINGS

FIG. 1: Changes in phage binding to S. thermophilus wild type strains and their phage-resistant mutants.

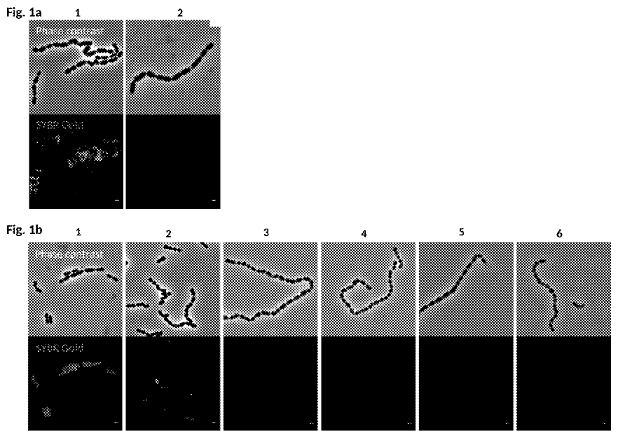

Phage DNA was labeled with SYBR Gold and visualized under green fluorescence. (a) Phage CHPC1057 adsorbs to its host STCH_09 (1) and it does not adsorb to STCH_09_BIM (2). (b) Phage CHPC926 adsorbs to its host STCH_15 (1), it has reduced adsorption with STCH_15_BIM_2 (2), and it does not adsorb to STCH_15_BIM_1, STCH_15_BIM_3, STCH_15_BIM_4, STCH_15_BIM_5 (panels no. 3-6, respectively). Scale bars: 1 μm.

FIG. 2: Fluorescence imaging of phage binding to S. thermophilus strains. (a) Adsorption of phages to their hosts was visualized with a conventional fluorescence microscope, after labeling phage DNA with SYBR Gold. (b) Super-resolution structured illumination microscopy (SRSIM) images of bacterial cells stained with Nile Red (red) and mixed with SYBR Gold DNA-labeled phages (green). Panels with phages and their host strains: (1) CHPC926 and STCH_15, (2) CHPC951 and STCH_12, (3) CHPC1057 and STCH_09, (4) CHPC1014 and STCH_13, (5) CHPC1046 and STCH_14. Two binding patterns are observed: spotty (panels no. 1, 2, 3) or diffused (panels no. 4, 5). Scale bars: 1 μm.

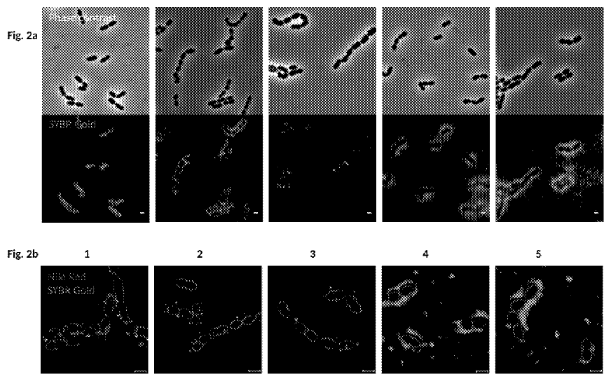

FIG. 3: Milk acidification of S. thermophilus STCH_15 and its derivatives in the presence and absence of phage CHPC926. (1) STCH_15_ΔepsE_1; (2) STCH_15_ΔepsE_1+CHPC926; (3) STCH_15_ΔepsE_2; (4) STCH_15_ΔepsE_2+CHPC926; (5) STCH_15; (6) STCH_15+CHPC926; (7) STCH_15. Samples 1, 2, 3, 4, 7 were supplemented with erythromycin (3 μg/ml). NC—negative control (milk).

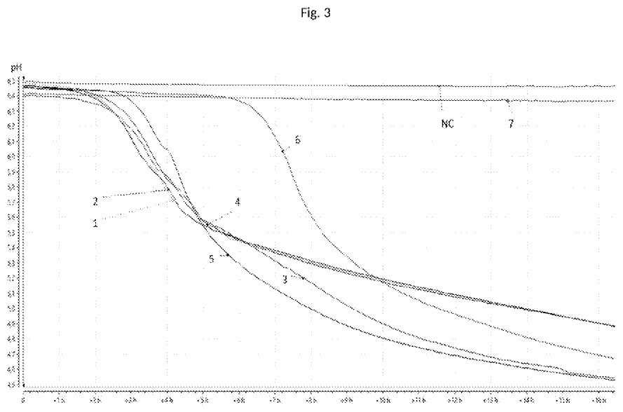

### DEPOSITS AND EXPERT SOLUTION

| Numbering herein | Deposit number |
|---|---|
| STCH_14 | DSM21408 |
| STCH_09 | DSM19243 |
| STCH_15 | DSM32842 |
| STCH_12 | DSM32826 |
| STCH_13 | DSM32841 |

The deposits were made according to the Budapest treaty on the international recognition of the deposit of microorganisms for the purposes of patent procedure.

The Applicant requests that a sample of the deposited microorganism should be made available only to an expert approved by the Applicant.

SEQUENCE LISTING
>STCH_09_epsH
ATGACAATCAGCATAGTAATCCCAGTTTATAATGTTCAAGATTACATAAA
AAAGTGTCTAGATTCTATATTAAGCCAGACATTTTCAGATTTAGAAATTA
TTCTTGTTGATGATGGTTCTACTGACTTGAGTGGAAGAATTTGTGATTAT
TATTCCGAAAATGATAAACGTATTAAAGTAATCCACACAGCAAATGGGGG
ACAGTCGGAAGCAAGGAACGTTGGAATCAAAAATGCCACATCAGAATGGA
TAACATTTATTGATTCTGATGACTACGTTTCTTCTGATTATATAGAGTAT
TTATATAATTTGATTCAAGTACACAATGCAGATATTTCAATAGCTAGTTT
TACCTATATCACACCTAAAAAGATAATTAAGCACGGTAACGGTGAAGTAG
CTCTTATGGATGCAAAAACTGCAATTCGGAGAATGTTACTGAATGAAGGT
TTCGATATGGGAGTTTGGGGGAAAATGTATCGAACGGAGTATTTTAATAA
ATATAAATTCGTTTCAGGAAAACTATTTGAAGATTCTTTAATTACATACC
AGATATTTTCAGAAGCTTCAACAATTGTTTTTGGAGCAAAGGATATTTAT
TTTTATGTTAACAGGAAAAATTCTACTGTTAATGGTACTTTTAATATAAA
AAAGTTTGATCTTATTGAAATGAATGAAGAAGCAAATAAGTTTATTAAAC
ATAAATTTCCAGATCTTTCATCTGAAGCACATCGTCGAATGATATGGGCA
TATTTTAGTACACTAAATCAAGTTTTATCATCAACTAATGAACACGATAT
TGATTTATATGCGCCACAATTAGTAGCTTATCTCCTTAAACAGGATAAAT
TCATAAAAAGGAATACTTTTATTCCCAAAAGAGATAAGATTGCATTTTTT
ATTTTAAAAAATTTTGGTTTAAAGACATATCGTAATGTTTGGAATTTATA
TTTAAAAATGACAAGATAA
>STCH_09_glycoslytransferase
ATGTCAGATTTACTCATTATTATTCCAGCCTATAACGAAGAAGGATCGAT
TGAAAATGTTGTTAACAACATCATTCAAAACTATCCTCAATATGACTATG
TTATCATCAATGATGGCTCTCGTGATAAAACTTCACAAATTTGCCATGAG
AATCATTACAATATTGTAGATTTACCAGTGAATCTCGGTCTTGCTGGGGC
TTTCCAAACAGGTTTAAGATATGCGTATGAGCATGGTTATAAAAAAGCGG
TGCAATTTGATGCCGATGGTCAACACTTACCAGAATATATTCAAAGTTTG
GAAGAAAAGATCGATGATGGTTTTGATCTAGTGATTGGTTCTCGATTTGT
CACAGAAAAACGACCAAATTCTTTACGAATGTTAGGCAATATCTTAATTA
GTTCAGCTATTAAACTCACTACTGGTAAGACAATTAAGGACCCAACTTCA
GGAATGAGAATGTTCTCAGAAGAGTTAATAAAAGAGTTTGCACTCAATAT
TAACTATGGTCCAGAGCCAGATACTGTTTCTTATCTCATCCGTAATGGTG
TAAAAGTTGCGGAAACACAAGTTAGAATGGAAGATAGACAGGCAGGAGAA
AGCTACTTGACTCTTTCTCGTTCGATTAAATATATGACACATATGTTTGT
GTCAATCCTACTCATTCAAAACTTTAGAAAGCGAGGCTAG
>STCH_13_epsD
ATGCCTTTATTAAAGTTAGTTAAATCAAAAGTAGACTTTGCTAAAAAGAC
GGAAGAGTATTATAACGCTATTCGCACAAATATTCAATTTTCTGGTGCTC
AGATTAAAGTGATTGCGATTAGCTCTGTTGAAGCTGGTGAAGGAAAATCA
ACGACATCTGTTAACTTGGCGATTTCATTTGCTAGTGTTGGGCTCCGAAC
ACTTCTGATTGATGCGGATACGCGTAACTCTGTTTTGTCAGGTACATTTA
AATCAAATGAGCCTTATAAAGGTCTTTCAAATTTCCTTTCAGGAAATGCC
GATCTAAATGAAACGATTTGCCAAACTGATATTTCTGGTTTAGATGTTAT
TTCATCTGGTCCTGTACCACCTAATCCAACAAGTCTTTTGCAAAATGACA
ATTTTAGACATTTGATGGAAGTTGCTCGTAGTCGTTATGATTATGTTATC
ATTGATACACCACCAATTGGTCTGGTCATTGATGCTGGTATTATTGCCCA
TCAGGCTGATGCTAGTCTTTTGGTTACAGCAGCTGGAAAAATTAAACGTC
GTTTCGTAACTAAGGCGGTCGAACAATTGAAACAAAGTGGTTCTCAGTTC
TTAGGTGTCGTCCTTAATAAAGTTGACATGACAGTTGATAAATATGGATC
ATATGGTTCTTACGGATCATATGGTGAGTACGGGAAAAAAACAGACCAAA
AAGAAGGTCATTCAAGAGCACATCGTCGTAGAAAAGGATAG
>STCH_14_epsE
GTGAAAGAAAAACAAGAAATTCGTCGCATTGAAATTGGTATTATACAGTT
GGTTGTGGTTGTTTTCGCAGCCATGGTAGCTAGTAAAATACCATATACAG
AGATTACCCAAGGAAGCATTGTCCTTTTAGGTGTCGTACATGTAGTGTCT
TACTATATCAGTAGTTATTATGAAAATCTTAAGTATAGAGGCTACTTGGA
TGAACTCATTGCAACTGTCAAATATTGTTTCATATTTGCTCTAATTGTAA
CATTTCTCTCGTTTTTTGCAGATGGAAGTTTTTCAATCTCACGTCGCGGA
CTTCTTTACGTCACCCTGATTTCAGGTGTTCTCTTATACGTTACAAATAC
TGTTCTTAAGTATTTCCGCTCATCTATTTATACACGTCGTAAAAGTAACA
AGAATATTCTCTTGATTTCTGATCAAGCACGTCTAGAAAATGTTTTGTCT
CGTATGAAAGACAATATGGATGGTAGGATTACAGCAGTTTGTGTCTTGGA
TAATCCTTATTTTACCGATCCATTTATCAAGAGTGTTAAACCTGAAAATT
TGATTGAATATGCGACACACTCAGTAGTAGACCAAGTTTTGATTAATCTG
CCAAGTGGGCAGTATAAGATTTGGGATTATGCATCACCTTTTGAGATCAT
GGGAATTCCAGTTTCTATTAATTTGAATGCCCTTGAATTTATGAATCAAG
GTGAAAAACGTATTCAACAATTGGGTCCTTTCAAAGTTGTTACGTTTTCA
ACGTATTTTTATAGCTATGGAGATATCTTGGCGAAACGTTTCCTCGATAT
CTGTGGAGCTCTAGTTGGTTTGTTGCTCTGTGGTATTGTAGGAATCTTCC
TTTATCCTCTTATTCGTAAAGATGGAGGACCAGCCATTTTTGCTCAAGAC
CGTGTGGGAGAAAATGGACGTATCTTCAAGTTTTATAAATTCCGTTCTAT
GTGTGTTGATGCGGAAGAAATCAAGAAGAATTTGATGGCACAGAATCAAA
TGTCTGGTGGTATGTTTAAGATGGACAATGATCCACGTATTACCAAAATT
GGACATTTCATTCGTAAAACGAGTCTTGATGAACTTCCACAATTTTGGAA
TGTTCTAAAAGGTGATATGAGCTTGGTTGGGACACGTCCTCCAACAGTTG
ATGAGTATGAAAAATATACACCTGAACAGAAACGTCGTTTAAGTTTTAAA
CCTGGTATCACTGGTCTTTGGCAAGTAAGCGGTCGAAGTGAAATTACTGA
TTTTGATGAAGTTGTAAAACTAGACGTTGCTTATTTGGACGGATGGACAA
TCTGGCGTGATATCAAAATCTTATTGAAAACGATTAAAGTAGTAGTAATG
AAGGATGGAGCAAAGTGA
>STCH_15_epsD_epsE
ATGCCTCTATTAAAGTTAGTAAAATCTAAAGTAAACTTTGCCAAACAAAC
AGAAGAGAATTACAATGCCATTCGCACAAATATTCAATTTTCTGGTGCTC
AGATTAAAGTGATTGCGATTAGCTCTGTTGAAGCTGGTGAAGGAAAATCA
ACGACATCTGTTAACTTGGCGATTTCATTTGCTAGTGTTGGGCTCCGAAC
ACTTCTGATTGATGCGGATACGCGTAATTCTGTTTTGTCAGGTACATTTA
AATCAAATGAGCCTTATAAAGGTCTTTCAAATTTCCTTTCAGGAAATGCC
GATCTAAATGAAACGATTTGCCAAACTGATATTTCTGGTTTAGATGTTAT
TGCATCTGGTCCTGTTCCACCTAATCCAACAAGTCTTTTGCAAAATGATA
ATTTTAGACATTTGATGGAAGTTGCTCGTAGTCGTTATGATTATGTCATC
ATCGATACACCACCTGTTGGGGTAGTTATTGATGCAGTTATTATTGCCCA
TCAGGCTGATGCTAGTCTTTTGGTTACAGAAGCTGGGAAAATCAAACGTC
GTTTCGTAACTAAGGCCGTTGAACAATTGGAACAAAGTGGTTCTCAGTTC
TTAGGGGTCGTCCTTAATAAAGTTGACATGACAGTTGATAAATATGGATC
GTATGGTTCTTACGGATCATATGGCGAGTATGGAAAAAAATCTGACCAAA
AAGAAGGTCATTCAAGAGCACATCGTCGTAGAAAAGGATGGCATTAATGG
GGATGATGCGGTTCCTTATAACCTTAACAGATTAAAAAGGGGTTTAGAGT
GAAAGAAAAACAAGAAATTCATCGCATTGAAATTGGTATTATACAGTTGG
TTGTGGTTGTTTTTGCAGCCATGATAGCTAGTAAAATACCTTATACAGAG
ATTACCCAAGGAAGCATTGTCCTTTTAGGTGTCATACATGTAGTGTCTTT
CTATATCAGTAGTTATTATGAAAATCTTAAGTATAGAGGCTACTTGGATG
AACTCATTGCAACTGTCAAATATTGTTTCATATTTGCTCTCATTGCAACA
TTTCTCTCGTTTTTTGCAGATGGAAGTTTTTCAATCTCACGTCGCGGACT
TCTTTACGTCACCCTGATTTCAGGTGTTCTCTTATATGTTACAAATACTG
TTCTTAAGTATTTCCGCTCATCTATTTATACACGTCGTAAAAGTAACAAG
AATATTCTCTTGATTTCTGATCAAGCACGTCTAGAAAATGTTTTGTCTCG
TATGAAAGACAATATGGATGGTAGGATTACAGCAGTTTGTGTCTTGGATA
ATCCTTATTTTACCGATCCATTTATCAAGAGTGTTAAACCTGAAAATTTG
ATTGAATATGCGACACACTCAGTAGTAGACCAAGTTTTGATTAATCTGCC
AAGTGGGCAGTATAAGATTTGGGATTATGCATCACCTTTTGAGATCATGG
GAATTCCAGTTTCTATTAATTTGAATGCCCTTGAATTTATGAGTCAAGGT
GAAAAACGTATTCAACAATTGGGTCCTTTCAAAGTTGTTACGTTTTCAAC
GCAATTTTATAGCTATGGAGATATCTTGGCGAAACGTTTCCTCGATATCT
GTGGAGCCCTAGTTGGTTTGGTGCTCTGTGGGATTGTTGGAATCTTCCTT
TATCCACTTATTCGTAAGGATGGTGGGCCAGCCATTTTTGCTCAAGACCG
TGTGGGAGAAAATGGACGTATCTTCAAGTTTTATAAATTCCGTTCTATGT
GTGTTGATGCGGAAGAAATCAAGAAGGATTTGATGGCACAGAATCAAATG
TCTGGTGGTATGTTTAAGATGGACAATGATCCACGTATTACCAAAATTGG
ACATTTCATTCGTAAAACGAGTCTTGATGAACTTCCACAATTTTGGAATG
TTCTAAAAGGTGATATGAGCTTGGTAGGAACACGTCCACCAACATTGGAT
GAGTACGAATCTTATACACCGGAACAAAAACGTCGCCTCAGCTTTAAACC
AGGTATTACTGGTCTTTGGCAAGTAAGCGGTCGAAGTGAAATTACTGATT
TTGATGAAGTTGTAAAACTAGACGTTGCTTATTTGGACGGATGGACAATC
TGGCGCGATATCAAAATCTTATTGAAAACAATTAAAGTAGTAGTAATGAA
GGATGGAGCAAAGTGATGGCTTTCACCATTTCTTTTAATGGTGATTAAAT
GACAAAAACAGTTTATAGCGTTTGTTCTAAGGGGATTCCAGCAAAATATG
GTGGATTTGAGACCTTTGTTGAGAAGTTGACAGAGTTCCAACAAGACAAA
GATATCCAATATTATGTAGCTTGTATGCGGGAAAACTCTGCAAAATCAGA
CATTACAGCAGATGTTTTTGAAGGTTCTGTTGCGAAGTTTGAAAATCAAA
CGCGCCCGCTCTGA
>STCH_15_epsK
ATGAAAATAGCATTAGTAGGTTCCAGCGGTGGCCATTTGACACACATGTA
TTTATTAAAAAAGTTTTGGGAAAATGAAGATAGATTTTGGGTCACATTTG
ATAAAACAGATGCAAAATCTATATTGAAAGAAGAAAGATTTTATCCTTGT
TATTATCCCACAAATAGAAATGTAAAAAACACGATAAAAAATACCATTCT
TGCATTTAAAATACTTAGAAAAGAAAAACCAGATTTGATTATTTCGAGTG
GTGCTGCGGTAGCCGTTCCTTTTTTTTGGTTAGGTAAACTATTCGGTGCA
AAGACAGTCTATATTGAAATATTTGACCGGATCGATAAACCAACCTTAAC
AGGAAAATTAGTTTATCCAGTTACTGATAAGTTTATAGTTCAATGGGAAG
AGTTAAAAAAAGTTTACCCTAAAGCGATTAATCTAGGAGGGATTTTCTAA

### REFERENCES

All references cited in this patent document are hereby incorporated herein in their entirety by reference.

1-17. (canceled)

18. A method of fermenting a milk substrate, comprising adding to the milk substrate an isolated bacteriophage insensitive mutant strain of the species Streptococcus thermophilus, wherein the mutant is derived from mother strain STCH_09 (deposited as DSM19243), STCH_12 (deposited as DSM32826), STCH_13 (deposited as DSM32841), STCH_14 (deposited as DSM21408), or STCH_15 (deposited as DSM32842), or a mutant or variant of any of these strains, wherein the mutant strain comprises a mutation in a gene involved in glycan biosynthesis, wherein the gene comprises a nucleotide sequence with at least 95% sequence identity to SEQ ID NO: 1, SEQ ID NO: 2, SEQ ID NO: 3, SEQ ID NO: 4, SEQ ID NO: 5, or SEQ ID NO: 6, wherein the mutation in the gene results in an impairment of glycan synthesis, and wherein the mutant strain is obtained by a method comprising:
(a) mutating a culture of a mother strain of Streptococcus thermophilus to obtain mutant strains of Streptococcus thermophilus, wherein the mother strain is STCH_09 (DSM19243), STCH_12 (DSM32826), STCH_13 (DSM32841), STCH_14 (DSM21408), or STCH_15 (DSM32842), or a mutant or variant of any of these strains;
(b) exposing the mutant strains to an isolated bacteriophage that attacks the mother strain; and
(c) selecting a bacteriophage insensitive mutant strain comprising a mutation in a gene comprising a nucleotide sequence with at least 95% sequence identity to SEQ ID NO: 1, SEQ ID NO: 2, SEQ ID NO: 3, SEQ ID NO: 4, SEQ ID NO: 5, or SEQ ID NO: 6, wherein said mutation results in an impairment of glycan synthesis,
and wherein the selected bacteriophage insensitive mutant strain shows increased robustness against phage attacks compared to its mother strain.

19. The method of claim 18, further comprising the step of deselecting mutant strains comprising a mutation in the CRISPR-region or a R/M region.

20. The method of claim 18, wherein the isolated bacteriophage insensitive mutant strain shows impaired attachment of phages to its cell surface compared to its mother strain.

21. The method of claim 18, wherein the isolated bacteriophage insensitive mutant strain shows reduced phage adsorption compared to its mother strain.

22. The method of claim 18, wherein the isolated bacteriophage insensitive mutant strain shows increased robustness against phage of the cos-, pac- or 987 type compared to its mother strain.

23. The method of claim 18, wherein the isolated bacteriophage insensitive mutant strain is a functionally equivalent mutant which has substantially the same properties as the mother strain, other than in the bacteriophage insensitivity.

24. The method of claim 18, wherein the isolated bacteriophage insensitive mutant strain is provided as a starter culture.

25. The method of claim 24, wherein the starter culture contains at least 10E8 CFU per gram the isolated bacteriophage insensitive mutant strain.

26. The method of claim 24, wherein the starter culture is in a frozen or dried form.

27. The method of claim 24, wherein the starter culture is freeze dried or spray dried.


SEQUENCE LISTING (6 sequences)

SEQ ID NO: 1 - DNA, 969 bp, Streptococcus thermophilus
  misc_feature 1..969: note = STCH_09_epsH
  source 1..969: mol_type = genomic DNA
atgacaatcagcatagtaatcccagtttataatgttcaagattacataaaaaagtgtctagattctatattaagccagacattttcagatttagaaattattcttgttgatgatggttctactgacttgagtggaagaatttgtgattattattccgaaaatgataaacgtattaaagtaatccacacagcaaatgggggacagtcggaagcaaggaacgttggaatcaaaaatgccacatcagaatggataacatttattgattctgatgactacgtttcttctgattatatagagtatttatataatttgattcaagtacacaatgcagatatttcaatagctagttttacctatatcacacctaaaaagataattaagcacggtaacggtgaagtagctcttatggatgcaaaaactgcaattcggagaatgttactgaatgaaggtttcgatatgggagtttgggggaaaatgtatcgaacggagtattttaataaatataaattcgtttcaggaaaactatttgaagattctttaattacataccagatattttcagaagcttcaacaattgtttttggagcaaaggatatttatttttatgttaacaggaaaaattctactgttaatggtacttttaatataaaaaagtttgatcttattgaaatgaatgaagaagcaaataagtttattaaacataaatttccagatctttcatctgaagcacatcgtcgaatgatatgggcatattttagtacactaaatcaagttttatcatcaactaatgaacacgatattgatttatatgcgccacaattagtagcttatctccttaaacaggataaattcataaaaaggaatacttttattcccaaaagagataagattgcattttttattttaaaaaattttggtttaaagacatatcgtaatgtttggaatttatatttaaaaatgacaagataa

SEQ ID NO: 2 - DNA, 690 bp, Streptococcus thermophilus
  misc_feature 1..690: note = STCH_09_glycoslytransferase
  source 1..690: mol_type = genomic DNA
atgtcagatttactcattattattccagcctataacgaagaaggatcgattgaaaatgttgttaacaacatcattcaaaactatcctcaatatgactatgttatcatcaatgatggctctcgtgataaaacttcacaaatttgccatgagaatcattacaatattgtagatttaccagtgaatctcggtcttgctggggctttccaaacaggtttaagatatgcgtatgagcatggttataaaaaagcggtgcaatttgatgccgatggtcaacacttaccagaatatattcaaagtttggaagaaaagatcgatgatggttttgatctagtgattggttctcgatttgtcacagaaaaacgaccaaattctttacgaatgttaggcaatatcttaattagttcagctattaaactcactactggtaagacaattaaggacccaacttcaggaatgagaatgttctcagaagagttaataaaagagtttgcactcaatattaactatggtccagagccagatactgtttcttatctcatccgtaatggtgtaaaagttgcggaaacacaagttagaatggaagatagacaggcaggagaaagctacttgactctttctcgttcgattaaatatatgacacatatgtttgtgtcaatcctactcattcaaaactttagaaagcgaggctag

SEQ ID NO: 3 - DNA, 741 bp, Streptococcus thermophilus
  misc_feature 1..741: note = STCH_13_epsD
  source 1..741: mol_type = genomic DNA
atgcctttattaaagttagttaaatcaaaagtagactttgctaaaaagacggaagagtattataacgctattcgcacaaatattcaattttctggtgctcagattaaagtgattgcgattagctctgttgaagctggtgaaggaaaatcaacgacatctgttaacttggcgatttcatttgctagtgttgggctccgaacacttctgattgatgcggatacgcgtaactctgttttgtcaggtacatttaaatcaaatgagccttataaaggtctttcaaatttcctttcaggaaatgccgatctaaatgaaacgatttgccaaactgatatttctggtttagatgttatttcatctggtcctgtaccacctaatccaacaagtcttttgcaaaatgacaattttagacatttgatggaagttgctcgtagtcgttatgattatgttatcattgatacaccaccaattggtctggtcattgatgctggtattattgcccatcaggctgatgctagtcttttggttacagcagctggaaaaattaaacgtcgtttcgtaactaaggcggtcgaacaattgaaacaaagtggttctcagttcttaggtgtcgtccttaataaagttgacatgacagttgataaatatggatcatatggttcttacggatcatatggtgagtacgggaaaaaaacagaccaaaaagaaggtcattcaagagcacatcgtcgtagaaaaggatag

SEQ ID NO: 4 - DNA, 1368 bp, Streptococcus thermophilus
  misc_feature 1..1368: note = STCH_14_epsE
  source 1..1368: mol_type = genomic DNA
gtgaaagaaaaacaagaaattcgtcgcattgaaattggtattatacagttggttgtggttgttttcgcagccatggtagctagtaaaataccatatacagagattacccaaggaagcattgtccttttaggtgtcgtacatgtagtgtcttactatatcagtagttattatgaaaatcttaagtatagaggctacttggatgaactcattgcaactgtcaaatattgtttcatatttgctctaattgtaacatttctctcgttttttgcagatggaagtttttcaatctcacgtcgcggacttctttacgtcaccctgatttcaggtgttctcttatacgttacaaatactgttcttaagtatttccgctcatctatttatacacgtcgtaaaagtaacaagaatattctcttgatttctgatcaagcacgtctagaaaatgttttgtctcgtatgaaagacaatatggatggtaggattacagcagtttgtgtcttggataatccttattttaccgatccatttatcaagagtgttaaacctgaaaatttgattgaatatgcgacacactcagtagtagaccaagttttgattaatctgccaagtgggcagtataagatttgggattatgcatcaccttttgagatcatgggaattccagtttctattaatttgaatgcccttgaatttatgaatcaaggtgaaaaacgtattcaacaattgggtcctttcaaagttgttacgttttcaacgtatttttatagctatggagatatcttggcgaaacgtttcctcgatatctgtggagctctagttggtttgttgctctgtggtattgtaggaatcttcctttatcctcttattcgtaaagatggaggaccagccatttttgctcaagaccgtgtgggagaaaatggacgtatcttcaagttttataaattccgttctatgtgtgttgatgcggaagaaatcaagaagaatttgatggcacagaatcaaatgtctggtggtatgtttaagatggacaatgatccacgtattaccaaaattggacatttcattcgtaaaacgagtcttgatgaacttccacaattttggaatgttctaaaaggtgatatgagcttggttgggacacgtcctccaacagttgatgagtatgaaaaatatacacctgaacagaaacgtcgtttaagttttaaacctggtatcactggtctttggcaagtaagcggtcgaagtgaaattactgattttgatgaagttgtaaaactagacgttgcttatttggacggatggacaatctggcgtgatatcaaaatcttattgaaaacgattaaagtagtagtaatgaaggatggagcaaagtga

SEQ ID NO: 5 - DNA, 2414 bp, Streptococcus thermophilus
  misc_feature 1..2414: note = STCH_15_epsD-epsE
  source 1..2414: mol_type = genomic DNA
atgcctctattaaagttagtaaaatctaaagtaaactttgccaaacaaacagaagagaattacaatgccattcgcacaaatattcaattttctggtgctcagattaaagtgattgcgattagctctgttgaagctggtgaaggaaaatcaacgacatctgttaacttggcgatttcatttgctagtgttgggctccgaacacttctgattgatgcggatacgcgtaattctgttttgtcaggtacatttaaatcaaatgagccttataaaggtctttcaaatttcctttcaggaaatgccgatctaaatgaaacgatttgccaaactgatatttctggtttagatgttattgcatctggtcctgttccacctaatccaacaagtcttttgcaaaatgataattttagacatttgatggaagttgctcgtagtcgttatgattatgtcatcatcgatacaccacctgttggggtagttattgatgcagttattattgcccatcaggctgatgctagtcttttggttacagaagctgggaaaatcaaacgtcgtttcgtaactaaggccgttgaacaattggaacaaagtggttctcagttcttaggggtcgtccttaataaagttgacatgacagttgataaatatggatcgtatggttcttacggatcatatggcgagtatggaaaaaaatctgaccaaaaagaaggtcattcaagagcacatcgtcgtagaaaaggatggcattaatggggatgatgcggttccttataaccttaacagattaaaaaggggtttagagtgaaagaaaaacaagaaattcatcgcattgaaattggtattatacagttggttgtggttgtttttgcagccatgatagctagtaaaataccttatacagagattacccaaggaagcattgtccttttaggtgtcatacatgtagtgtctttctatatcagtagttattatgaaaatcttaagtatagaggctacttggatgaactcattgcaactgtcaaatattgtttcatatttgctctcattgcaacatttctctcgttttttgcagatggaagtttttcaatctcacgtcgcggacttctttacgtcaccctgatttcaggtgttctcttatatgttacaaatactgttcttaagtatttccgctcatctatttatacacgtcgtaaaagtaacaagaatattctcttgatttctgatcaagcacgtctagaaaatgttttgtctcgtatgaaagacaatatggatggtaggattacagcagtttgtgtcttggataatccttattttaccgatccatttatcaagagtgttaaacctgaaaatttgattgaatatgcgacacactcagtagtagaccaagttttgattaatctgccaagtgggcagtataagatttgggattatgcatcaccttttgagatcatgggaattccagtttctattaatttgaatgcccttgaatttatgagtcaaggtgaaaaacgtattcaacaattgggtcctttcaaagttgttacgttttcaacgcaattttatagctatggagatatcttggcgaaacgtttcctcgatatctgtggagccctagttggtttggtgctctgtgggattgttggaatcttcctttatccacttattcgtaaggatggtgggccagccatttttgctcaagaccgtgtgggagaaaatggacgtatcttcaagttttataaattccgttctatgtgtgttgatgcggaagaaatcaagaaggatttgatggcacagaatcaaatgtctggtggtatgtttaagatggacaatgatccacgtattaccaaaattggacatttcattcgtaaaacgagtcttgatgaacttccacaattttggaatgttctaaaaggtgatatgagcttggtaggaacacgtccaccaacattggatgagtacgaatcttatacaccggaacaaaaacgtcgcctcagctttaaaccaggtattactggtctttggcaagtaagcggtcgaagtgaaattactgattttgatgaagttgtaaaactagacgttgcttatttggacggatggacaatctggcgcgatatcaaaatcttattgaaaacaattaaagtagtagtaatgaaggatggagcaaagtgatggctttcaccatttcttttaatggtgattaaatgacaaaaacagtttatagcgtttgttctaaggggattccagcaaaatatggtggatttgagacctttgttgagaagttgacagagttccaacaagacaaagatatccaatattatgtagcttgtatgcgggaaaactctgcaaaatcagacattacagcagatgtttttgaaggttctgttgcgaagtttgaaaatcaaacgcgcccgctctga

SEQ ID NO: 6 - DNA, 450 bp, Streptococcus thermophilus
  misc_feature 1..450: note = STCH_15_epsK
  source 1..450: mol_type = genomic DNA
atgaaaatagcattagtaggttccagcggtggccatttgacacacatgtatttattaaaaaagttttgggaaaatgaagatagattttgggtcacatttgataaaacagatgcaaaatctatattgaaagaagaaagattttatccttgttattatcccacaaatagaaatgtaaaaaacacgataaaaaataccattcttgcatttaaaatacttagaaaagaaaaaccagatttgattatttcgagtggtgctgcggtagccgttccttttttttggttaggtaaactattcggtgcaaagacagtctatattgaaatatttgaccggatcgataaaccaaccttaacaggaaaattagtttatccagttactgataagtttatagttcaatgggaagagttaaaaaaagtttaccctaaagcgattaatctaggagggattttctaa